(19) United States
(12) Patent Application Publication
(10) Pub. No.: US 2004/0223992 A1
(43) Pub. Date: Nov. 11, 2004

(54) Wet skin treatment compositions comprising gel-networks

(75) Inventors: Mannie Lee Clapp, Mason, OH (US); Rebecca Ann Taylor, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/841,195

(22) Filed: May 7, 2004

Related U.S. Application Data
(60) Provisional application No. 60/469,552, filed on May 9, 2003.
(60) Provisional application No. 60/515,030, filed on Oct. 28, 2003.

(51) Int. Cl. A61K 9/14; A61K 7/00

(52) U.S. Cl. 424/401

(57) ABSTRACT

The present invention relates to a personal care composition containing a gel-network phase, a stability agent, a structured oil phase and an aqueous phase. This particular combination of materials, specifically gel-network emulsifiers in combination with stabilizing polymers, provide for products which have excellent application aesthetics and yet deposit significant levels of beneficial lipids even when rinsed off. Additionally, the present invention relates to a method of using a wet skin personal care composition that contains a gel-network phase, a stability agent, a structured oil phase and an aqueous phase.

### CROSS REFERENCE TO RELATED APPLICATIONS

This application claims the benefit of Provisional application Ser. No. 60/469,552, filed May 9, 2003, and of Provisional application Ser. No. 60/515,030, filed Oct. 28, 2003.

### TECHNICAL FIELD

The present invention relates to the field of wet skin treatment compositions for improving skin feel of keratinous surfaces. More specifically, rinsable wet skin treatment compositions are provided that provide excellent skin moisturization and conditioning.

### BACKGROUND OF THE INVENTION

Wet skin treatment compositions are well known and widely used. These compositions have long been employed to cleanse and moisturize skin, deliver actives, hide imperfections and to reduce the oiliness/shine associated with sebum

Skin conditioning compositions that provide moisturizing benefits are known. Many of these compositions are aqueous systems comprising an emulsified conditioning oil or other similar material stabilized with surfactant. Typically, skin moisturizing compositions are in the form of lotions meant to be applied to the skin after bathing and throughout the day if reapplication is necessary.

Skin is made up of several layers of cells, which coat and protect the keratin and collagen fibrous proteins that form the skeleton of its structure. The outermost of these layers, referred to as the stratum corneum, is known to be composed of 25 nm protein bundles surrounded by 8 nm thick layers. Anionic surfactants and organic solvents typically penetrate the stratum corneum membrane and, by delipidization (i.e. removal of the lipids from the stratum corneum), destroy its integrity. This destruction of the skin surface topography leads to a rough feel and may eventually permit the surfactant or solvent to interact with the keratin, creating irritation.

It is now recognized that maintaining the proper water gradient across the stratum corneum is important to its functionality. Most of this water, which is sometimes considered to be the stratum corneum's plasticizer, comes from inside the body. If the humidity is too low, such as in a cold climate, insufficient water remains in the outer layers of the stratum corneum to properly plasticize the tissue, and the skin begins to scale and becomes itchy. Skin permeability is also decreased somewhat when there is inadequate water across the stratum corneum. On the other hand, exposure to high water concentration for long periods of time on the outside of the skin causes the stratum corneum to ultimately absorb three to five times its own weight of bound water. This swells and puckers the skin and results in approximately a two to three fold increase in the permeability of the skin to water and moisturizer molecules. In the shower or bath, as skin becomes hydrated, this is recognized as an ideal time to deliver moisturizer to the skin since absorption of the moisturizer will be high.

It is further desirable to deliver the above skin conditioning benefits via an in-the-shower or in-the-bath lotion. Unfortunately, in the shower/bath, moisturizers are often readily rinsed from the skin. This is particularly true when surfactant is present.

Thus, a need still exists for compositions which will effectively deposit moisturizers and/or other skin benefit agents in the shower and/or bath and thereby assist the stratum corneum in maintaining its barrier and water-retention functions at optimum washing.

### SUMMARY OF THE INVENTION

The inventors have discovered that rinsable wet skin treatment compositions comprising emulsifiers, polymeric stabilizers, lipid and an aqueous phase will effectively deposit oils and/or other skin benefit agents in the shower and/or bath. The inventors have found that particular combinations of materials, specifically non-ionic gel-network emulsifiers in combination with stabilizing polymers, provide for products which have excellent application aesthetics and yet deposit significant levels of beneficial lipids even when rinsed off. Additionally, the compositions provide softer skin feel across all skin types and at the same time assist the stratum corneum in maintaining its barrier and water-retention functions at optimum performance in spite of deleterious interactions which the skin may encounter in washing, work, and recreation. These compositions provide improved skin appearance, aesthetics and skin feel during and/or after application, and are especially useful in providing improved deposition or effectiveness of skin conditioning agents to the desired area of the skin.

### DETAILED DESCRIPTION OF THE INVENTION

All percentages and ratios used herein are by weight of the total composition and all measurements made are at 25° C., unless otherwise designated.

The compositions of the present invention can comprise, consist essentially of, or consist of, the essential as well as optional ingredients and components described herein. As used herein, “consisting essentially of” means that the composition or component may include additional ingredients, but only if the additional ingredients do not materially alter the basic and novel characteristics of the claimed compositions or methods.

All publications cited herein are hereby incorporated by reference in their entirety.

The term “topical application”, as used herein, means to apply or spread the compositions of the present invention onto the surface of the skin.

The term “dermatologically-acceptable,” as used herein, means that the compositions or components thereof so described are suitable for use in contact with human skin without undue toxicity, incompatibility, instability, allergic response, and the like.

The term “safe and effective amount” as used herein means an amount of a compound, component, or composition sufficient to significantly induce a positive benefit, preferably a positive skin appearance or feel benefit, including independently the benefits disclosed herein, but low enough to avoid serious side effects, i.e., to provide a reasonable benefit to risk ratio, within the scope of sound medical judgment.

Active and other ingredients useful herein may be categorized or described herein by their cosmetic and/or therapeutic benefit or their postulated mode of action. However, it is to be understood that the active and other ingredients useful herein can in some instances provide more than one cosmetic and/or therapeutic benefit or operate via more than one mode of action. Therefore, classifications herein are made for the sake of convenience and are not intended to limit an ingredient to the particularly stated application or applications listed.

The compositions of the invention are useful for topical application and for providing an essentially immediate (i.e., acute) skin feel following rinse off of the composition to on the keratinous surface. Without intending to be limited by theory, it is believed that this acute skin feel improvement results at least in part from therapeutic coverage or masking of skin imperfections by the deposition of oil.

More particularly, the compositions of the present invention are useful for regulating skin condition, including regulating visible and/or tactile discontinuities in skin, including but not limited to visible and/or tactile discontinuities in skin texture and/or color, more especially discontinuities associated with skin aging. Such discontinuities may be induced or caused by internal and/or external factors. Extrinsic factors include ultraviolet radiation (e.g., from sun exposure), environmental pollution, wind, heat, low humidity, harsh surfactants, abrasives, and the like. Intrinsic factors include chronological aging and other biochemical changes from within the skin.

Aqueous Phase

The continuous aqueous phase generally comprises from no more than about 90 weight percent of a fluid, preferably no more than about 80 weight percent, even more preferably no more than about 70 weight percent, still more preferably no more than about 60 weight percent. The continuous aqueous phase of the present invention typically comprises at least 10 weight percent of a fluid, preferably at least 20 weight percent, even more preferably at least 30 weight percent, still more at least 40 weight percent of a fluid. The aqueous phase is the continuous phase of the instant composition in which the structured oil phase is dispersed. The aqueous phase contains the dispersion stabilizer, and optionally such ingredients as preservatives, wetting agents, auxiliary emulsifiers and various optional benefit agents.

Structured Oil Phase

The structured oil phase comprises two essential components: a skin compatible oil, and a structurant that can form a stable network at a temperature below 35° C.

Skin Compatible Oil

A skin compatible oil is defined here, as an oil that is liquid or semi-solid at the temperature at which bathing is carried out that is deemed safe for use in cosmetics being either inert to the skin or actually beneficial. The composition comprises no more than 80 weight percent of said skin compatible oil, preferably no more than 70 weight percent, still more preferably no more than 60 weight percent, and most preferably no more than 50 weight percent of the skin compatible oil. The composition comprises at least 1 weight percent, preferably at least 5 weight percent, even more preferably at least 7 weight percent, and most preferably at least 10 weight percent of the skin compatible oil. The most useful skin compatible oils for the present invention include ester oils, hydrocarbon oils, and silicone oils.

Ester oils, as the name implies, have at least one ester group in the molecule. One type of common ester oil useful in the present invention are the fatty acid mono and polyesters such as cetyl octanoate, octyl isonanoanate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate; sucrose ester and polyesters, sorbitol ester, and the like.

A second type of useful ester oil is predominantly comprised of triglycerides and modified triglycerides. These include vegetable oils such as jojoba, soybean, canola, sunflower, safflower, rice bran, avocado, almond, olive, sesame, persic, castor, coconut, and mink oils. Synthetic triglycerides can also be employed provided they are liquid at room temperature. Modified triglycerides include materials such as ethoxylated and maleated triglyceride derivatives provided they are liquids. Proprietary ester blends such as those sold by Finetex as Finsolv are also suitable, as is ethylhexanoic acid glyceride.

A third type of ester oil is liquid polyester formed from the reaction of a dicarboxylic acid and a diol. Examples of polyesters suitable for the present invention is the polyesters marketed by ExxonMobil under the trade name PURESYN ESTER.R™.

A second class of skin compatible oils suitable for the present invention is liquid and semi-solid hydrocarbons. These include linear and branched oils such as liquid paraffin, squalene, squalane, mineral oil, low viscosity synthetic hydrocarbons such as polyalphaolefin sold by ExxonMobil under the trade name of PURESYN PAO and polybutene under the trade name PANALANE or INDOPOL. Light (low viscosity) highly branched hydrocarbon oils are also suitable.

Petrolatum is a unique hydrocarbon material and a useful component of the present invention. Its semi-solid nature can be controlled both in production and by the formulator through blending with other oils. Since it is only partially comprised of a liquid fraction at room temperature, it is more properly regarded as either the “structured oil phase” when present by itself or alternatively as the “structurant” when admixed with other skin compatible oils.

A third class of useful skin compatible oils is silicone based. They include linear and cyclic polydimethyl siloxane, organo functional silicones (alkyl and alkyl aryl), and amino silicones.

Structurant

The second component of the structured oil phase is a structurant. The structurant must satisfy two requirements. Firstly, the structurant must be capable of forming a stable network of in the skin compatible oil phase at a temperature below 35° C. This property is critical so that the structured oil is active during use but is not perceived as gritty. By stable, we mean the network survives at least one month of storage at 25° C. and 35° C.

The second requirement is that the structurant provides structured oil phase with the correct rhelogical properties. The structured oil phase should have a viscosity in the range of 100 to about 200,000 poise measured at 1 Sec−1, preferably 200 to about 100,000 poise, and most preferably 200 to about 50,000 poise as determined using the lipid rheology method described below. The amount of structurant required to produce this viscosity will vary depending on the oil and the structurant, but in general, the structurant will preferably be no more than about 75 weight percent of the structured oil phase, more preferably no more than about 50 weight percent, and still more preferably no more than about 35 weight percent of the structured oil phase. Structurants meeting the above requirements with the selected skin compatible oil can form 3-dimensional network to build up the viscosity of the selected oils. It has been found that such structured oil phases, i.e., built with the 3-dimensional network, are extremely desirable for use as wet-skin treatment compositions used in bathing. These structured oils can deposit and be retained very effectively on wet skin and retained after rinsing and drying to provide long-lasting after wash skin benefit without causing a too oily/greasy wet and dry feel. It is believed that the highly desirable in-use and after-use properties of such structured oils are due to their shear thinning rheological properties and the weak structure of the network. Due to its high low-shear viscosity, the solid-network structured oil can stick and retain well on the skin during application of the skin conditioner. After being deposited on the skin, the network yields easily during rubbing due to the weak structuring of the crystal network and its lower high-shear viscosity.

The degree of shear-thinning (which is described in the Lipid Rheology Method described herein) exhibited by the structured oil phase is given by the value of n from the Power Law Model. Newtonian fluids which exhibit no shear thinning properties have n values close to one, while lower values indicate that the structured oil phase is more shear-thinning. For the present invention, it is preferred that the structured oil phase have a shear index less than 0.8, more preferably less than 0.6, even more preferably less than 0.5 and most preferably less than 0.4.

The structurant can be an organic structurant that is either crystalline solids or amorphous gels with molecular weight less than 5,000 Daltons, preferably less than 3,000 Daltons. Preferred organic structurants have a melting point greater than 35° C., preferably greater than 40° C. Especially preferred structurants are those that can form a solution with the selected skin compatible oil at a temperature higher than their melting point to form a free flowing clear solution. Upon cooling to the ambient temperature, the organic structurant precipitate from the oil phase to form a 3-dimensional structure providing the physical properties set forth above. Examples of organic thickeners suitable for the invention are solid fatty acid esters, natural or modified fats, fatty acid, fatty amine, fatty alcohol, natural and synthetic waxes, and petrolatum. Petrolatum is a preferred organic structuring agents.

Particularly preferred organic structurants are solid fatty acid esters and petrolatum. Examples of solid fatty esters are mono, di or tri glycerides derivatives of palmitic acid, stearic acid, or hydroxystearic acid; sugar fatty ester or fatty esters of dextrin. Examples of these polyol fatty acid esters are described in U.S. Pat. Nos. 5,427,704, 5,472,728, 6156369, 5490995 and EP patent 398409 incorporated by reference herein. Trihydroxystearin sold under the trade name of THIXCIN R from Rheox Corporation is found particularly useful for structuring triglyceride ester oils.

Inorganic structuring agents include hydrophobically modified silica or hydrophobically modified clay. Nonlimiting examples of inorganic structurants are BENTONE 27V, BENTONE 38V or BENTONE GEL MIO V from Rheox; and CAB-O-SIL TS720 or CAB-O-SIL M5 from Cabot Corporation.

The level of structurant present in the structured oil phase can be in the range of 0 to 90% and depends on the type of structurant used and the nature of the skin compatible oil. For solid organic structurants such as trihydroxystearin, the preferred level is 3 to 15%. However, the exact levels used should provide a stable network having the desired viscosity.

Gel-Network

The ‘gel-network’ of the present invention is composed of a hydrophobic structuring agent and a non-ionic, hydrophilic surfactant. Preferred levels of these individual components are specified below, however the total gel-network portion of the composition is limited separately from its individual components. Without being bound by theory, it is believed that the gel-network allows for good application of the product in the wet environment of bathing. As the product is applied it is diluted with the water present on the skin and possibly that of the bath or shower. The gel-network allows for a ‘smooth’ dilution of the product, allowing the product to be spread easily and deposit evenly on the skin of the user. However, higher levels of gel-network interfere with deposition, effectively releasing less lipid from the composition and result in more rinse-off, or lower deposition efficiency. For this reason, in the present invention the level of gel-network in the product is preferably kept relatively low. In a preferred embodiment, the gel network will not form a homogeneous aqueous phase as described in the gel-network stability test. Without the gel-network in the product, i.e. a product consisting essentially of water, aqueous phase stability agent and lipid, application characteristics are undesirable and the product is difficult to spread and deposit evenly.

The present invention comprises no more than about 20 weight percent, preferably no more than about 10 weight percent, and more preferably no more than about 5 weight percent, of a hydrophobic, structuring agent selected from the group consisting of saturated C₁₆ to C₃₀ fatty alcohols, saturated C₁₆ to C₃₀ fatty alcohols containing from about 1 to about 5 moles of ethylene oxide, saturated C₁₆ to C₃₀diols, saturated C₁₆ to C₃₀ monoglycerol ethers, saturated C₁₆ to C₃₀ hydroxy fatty acids, and mixtures thereof, having a melting point of at least about 40° C. The present invention preferably comprises at least 0.5 weight percent, more preferably at least 1 weight percent, even more preferably at least 2 weight percent, and still more preferably at least 3 weight percent, of a hydrophobic, structuring agent selected from the group consisting of saturated C₁₆ to C₃₀ fatty alcohols, saturated C₁₆ to C₃₀ fatty alcohols containing from about 1 to about 5 moles of ethylene oxide, saturated C₁₆ to C₃₀diols, saturated C₁₆ to C₃₀ monoglycerol ethers, saturated C₁₆ to C₃₀ hydroxy fatty acids, and mixtures thereof, having a melting point of at least about 40° C. Without being limited by theory, it is believed that these structuring agents are useful to assist in the formation of the rheological characteristic of the composition which contribute to the hydrolytic stability of the composition of the present invention. In particular structuring agents assist in the formation of the liquid crystalline gel network structures.

The preferred structuring agents of the present invention are selected from the group consisting of stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, and mixtures thereof. More preferred structuring agents of the present invention are selected from the group consisting of stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof. Even more preferred structuring agents are selected from the group consisting of stearyl alcohol, cetyl alcohol, behenyl alcohol, steareth-2, and mixtures thereof.

Hydrophilic Surfactant

The compositions of the present invention comprise no more than about 10 weight percent, preferably no more than about 6 weight percent, and more preferably no more than about 3% of at least one hydrophilic surfactant. The compositions of the present invention comprise at least 0.1 weight percent, preferably at least 0.2 weight percent, and more preferably at least 0.3 weight percent of at least one hydrophilic surfactant. Without being limited by theory, it is believed that the hydrophilic surfactant disperses the hydrophobic materials, i.e. the structuring agent, in the water phase. The surfactant, at a minimum, must be hydrophilic enough to disperse in water.

The exact surfactant chosen will depend upon the pH of the composition and the other components present. Preferred for use herein are nonionic surfactants. Among the nonionic surfactants that are useful herein are those that can be broadly defined as condensation products of long chain alcohols, e.g. C8-30 alcohols, with sugar or starch polymers, i.e., glycosides. These compounds can be represented by the formula (S).ₙ—O—R wherein S is a sugar moiety such as glucose, fructose, mannose, and galactose; n is an integer of from about 1 to about 1000, and R is a C8-30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants include those wherein S is a glucose moiety, R is a C8-20 alkyl group, and n is an integer of from about 1 to about 9. Commercially available examples of these surfactants include decyl polyglucoside (available as APG 325 CS from Henkel) and lauryl polyglucoside (available as APG 600 CS and 625 CS from Henkel).

Other useful non-ionic surfactants included the condensation products of sorbitol with a fatty acid. Nonlimiting examples include the Tweens, Spans, and the Polysorbates.

Other useful nonionic surfactants include the condensation products of alkylene oxides with fatty acids (i.e. alkylene oxide esters of fatty acids). These materials have the general formula RCO(X).ₙ.OH wherein R is a C10-30 alkyl group, X is —OCH₂CH₂— (i.e. derived from ethylene glycol or oxide) or —OCH₂CHCH₃— (i.e. derived from propylene glycol or oxide), and n is an integer from about 6 to about 100. Other nonionic surfactants are the condensation products of alkylene oxides with 2 moles of fatty acids (i.e. alkylene oxide diesters of fatty acids). These materials have the general formula RCO(X).ₙOOCR wherein R is a C10-30 alkyl group, X is —OCH₂CH₂— (i.e. derived from ethylene glycol or oxide) or —OCH₂CHCH₃—(i.e. derived from propylene glycol or oxide), and n is an integer from about 6 to about 100. Other nonionic surfactants are the condensation products of alkylene oxides with fatty alcohols (i.e. alkylene oxide ethers of fatty alcohols). These materials have the general formula R(X)ₙOR′ wherein R is a C10-30 alkyl group, X is —OCH₂CH₂— (i.e. derived from ethylene glycol or oxide) or —OCH₂CHCH₃— (i.e. derived from propylene glycol or oxide), and n is an integer from about 6 to about 100 and R′ is H or a C10-30 alkyl group. Still other nonionic surfactants are the condensation products of alkylene oxides with both fatty acids and fatty alcohols >i.e. wherein the polyalkylene oxide portion is esterified on one end with a fatty acid and etherified (i.e. connected via an ether linkage) on the other end with a fatty alcohol. These materials have the general formula RCO(X)ₙOR′ wherein R and R′ are C10-30 alkyl groups, X is —OCH₂CH₂(i.e. derived from ethylene glycol or oxide) or —OCH₂CHCH₃— (derived from propylene glycol or oxide), and n is an integer from about 6 to about 100. Nonlimiting examples of these alkylene oxide derived nonionic surfactants include ceteth-6, ceteth-10, ceteth-12, ceteareth-6, ceteareth-10, ceteareth-12, steareth-6, steareth-10, steareth-12, PEG-6 stearate, PEG-10 stearate, PEG-12 stearate, PEG-20 glyceryl stearate, PEG-80 glyceryl tallowate, PPG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof.

Still other useful nonionic surfactants include polyhydroxy fatty acid amide surfactants An especially preferred surfactant corresponding to the above structure is coconut alkyl N-methyl glucoside amide. Processes for making compositions containing polyhydroxy fatty acid amides are disclosed, for example, in G. B. Patent Specification 809,060, published Feb. 18, 1959, by Thomas Hedley & Co., Ltd.; U.S. Pat. No. 2,965,576, to E. R. Wilson, issued Dec. 20, 1960; U.S. Pat. No. 2,703,798, to A. M. Schwartz, issued Mar. 8, 1955; and U.S. Pat. No. 1,985,424, to Piggott, issued Dec. 25, 1934; which are incorporated herein by reference in their entirety.

Preferred among the nonionic surfactants are those selected from the group consisting of steareth-21, ceteareth-20, ceteareth-12, Tween-60, Tween-80, sucrose cocoate, steareth-100, PEG-100 stearate, PEG-1000 stearate, and mixtures thereof.

Emulsifier Systems

In addition, there are several commercial emulsifier mixtures that are useful in some embodiments. Nonlimiting examples include PROLIPID 141 (glyceryl stearate, behenyl alcohol, palmitic acid, stearic acid, lecithin, lauryl alcohol, myristyl alcohol and cetyl alcohol) and 151 (Glyceryl stearate, cetearyl alcohol, stearic acid, 1-propanamium, 3-amino-N-(2-(hydroxyethyl)-N-N-Dimethyl,N-C(16-18) Acyl Derivatives, Chlorides) from ISP; POLAWAX NF (Emulsifying wax NF), from Croda; and EMULLIUM DELTA (cetyl alcohol, glyceryl stearate, peg-75 stearate, ceteth-20 and steareth-20) from Gattefosse.

Preferably the composition has a total flash lather volume less than 100 ml, more preferably less than 50 ml and even more preferably less than 25 ml as described in the Lather Volume Test. Ideal compositions are effectively ‘non-lathering’

Polymeric Stabilizer

The compositions of the present invention, include one or more polymeric stabilizer. The stability of the product is dependent upon the polymeric stabilizer as the non-ionic gel network phase is intentionally kept so low as to intentionally be unstable if used singly. Because stabilizers thicken with different efficiencies, it is difficult to provide an accurate compositional range, however, when present, the composition preferably comprises no more than about 10 weight percent, more preferably no more than 8 weight percent, and still more preferably no more than 7 weight percent, by weight of the composition of the polymeric stabilizer. When present, the composition preferably comprises at least 0.01 weight percent, more preferably at least 0.05 weight perent, and still more preferably at least 0.1 weight percent, by weight of the composition of the polymeric stabilizer. A better method of describing the Polymer Stabilizer is to say that it must build viscosity in the product. This can be measured using the Polymeric Stabilizer Viscosity Test. Preferably, the stability agent produces a viscosity in this test of at least 1000 cps, more preferably at least 1500 cps, and still more preferably at least 2000 cps.

Nonlimiting examples of polymeric stabilizers useful herein include carboxylic acid polymers such as the carbomers (such as those commercially available under the tradename CARBOPOL® 900 series from B.F. Goodrich; e.g., CARBOPOL® 954). Other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as CARBOPOL® 1342, CARBOPOL® 1382, PEMULEN TR-1, and PEMULEN TR-2, from B.F. Goodrich.

Other nonlimiting examples of polymeric stabilizers include crosslinked polyacrylate polymers including both cationic and nonionic polymers.

Still other nonlimiting examples of polymeric stabilizers include the polyacrylamide polymers, especially nonionic polyacrylamide polymers including substituted branched or unbranched polymers. More preferred among these polyacrylamide polymers is the nonionic polymer given the CTFA designation polyacrylamide and isoparaffin and laureth-7, available under the Tradename SEPIGEL 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500W, SSSA100H, from Lipo Chemicals, Inc., (Patterson, N.J.).

Another nonlimiting class of polymeric stabilizers useful herein are the polysaccharides. Nonlimiting examples of polysaccharide gelling agents include those selected from cellulose, and cellulose derivatives. Preferred among the alkyl hydroxyalkyl cellulose ethers is the material given the CTFA designation cetyl hydroxyethylcellulose, which is the ether of cetyl alcohol and hydroxyethylcellulose, sold under the tradename NATROSEL® CS PLUS from Aqualon Corporation (Wilmington, Del.). Other useful polysaccharides include scleroglucans which are a linear chain of (1-3) linked glucose units with a (1-6) linked glucose every three units, a commercially available example of which is CLEAROGEL™ CS11 from Michel Mercier Products Inc. (Mountainside, N.J.).

Another nonlimiting class of polymeric stabilizers useful herein are the gums. Nonlimiting examples of gums useful herein, xantham gum gellan gum, and mixtures thereof.

Yet another nonlimiting class of polymeric stabilizers useful herein are the modified starches. Acrylate modified starches such as WATERLOCK® from Grain Processing Corporation may be used. Hydroxypropyl starch phosphate, tradename STRUCTURE XL from National Starch is another example of a useful modified starch, and other useful examples include ARISTOFLEX HMB (Ammonium Acrylodimethyltaruate/Beheneth-25 Methacrylate Crosspolymer) from Clariant. Preferred among the polymeric stabilizers are the modified starches such as structure XL and its mixtures with other polymeric stabilizers.

Optional Ingredients

The compositions of the present invention may contain one or more additional skin care components. In a preferred embodiment, where the composition is to be in contact with human keratinous tissue, the additional components should be suitable for application to keratinous tissue, that is, when incorporated into the composition they are suitable for use in contact with human keratinous tissue without undue toxicity, incompatibility, instability, allergic response, and the like within the scope of sound medical judgment.

The CTFA Cosmetic Ingredient Handbook, Second Edition (1992) describes a wide variety of nonlimiting cosmetic and pharmaceutical ingredients commonly used in the personal care industry, which are suitable for use in the compositions of the present invention.

In any embodiment of the present invention, however, the additional components useful herein can be categorized by the benefit they provide or by their postulated mode of action. However, it is to be understood that the additional components useful herein can in some instances provide more than one benefit or operate via more than one mode of action. Therefore, classifications herein are made for the sake of convenience and are not intended to limit the active to that particular application or applications listed.

Shiny Particles

Nonlimiting examples of the interference pigments useful herein include those supplied by Persperse, Inc. under the trade name PRESTIGE®, FLONAC®; supplied by EMD Chemicals, Inc. under the trade name TIMIRON®, COLORONA®, DICHRONA® and XIRONA®; and supplied by Engelhard Co. under the trade name FLAMENCO®, TIMICA®, DUOCHROME®.

A second class of interference pigment is based on cholesteric liquid crystal, e.g. HELICONE® HC supplied by KOBO products. HELICONE® HC is composed of transparent platelets of polyacrylates with a helical superstructure. As part of this structure, cigar-shaped liquid crystal molecules are fixed into layers of parallel rows. Each layer has a slightly different molecular orientation and the distance between two layers with the same molecular orientation defines as the “pitch”, which determines the color. This type pigment is hydrophobic. Therefore, they can be used without surface treatment

Other Optional Ingredients

Other non limiting examples of optional ingredients include benefit agents that are selected from the group consisting of vitamins and derivatives thereof (e.g., ascorbic acid, vitamin E, tocopheryl acetate, and the like); sunscreens; thickening agents (e.g., polyol alkoxy ester, available as CROTHIX from Croda); preservatives for maintaining the anti microbial integrity of the cleansing compositions; anti-acne medicaments (resorcinol, salicylic acid, and the like); antioxidants; skin soothing and healing agents such as aloe vera extract, allantoin and the like; chelators and sequestrants; and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol), antibacterial agents and mixtures thereof. These materials can be used at ranges sufficient to provide the required benefit, as would be obvious to one skilled in the art.

Analytical Methods

Lipid Rheology Test

Lipid rheology is measured on a TA Instruments AR2000 stress-controlled rheometer with a Peltier temperature controlled sample stage or an equivalent. A parallel plate geometry is used with a 40 mm plate and a 1 mm gap. The lower plate is heated to 85° C. and the melted lipid and structurant (if present) is added onto the lower plate and allowed to equilibrate. The upper plate is then lowered to the 1 mm gap while ensuring the lipid fills the gap fully, spinning the top plate and adding more lipid to promote wicking, and the sample is cooled quickly to 25° C. and equilibrated at 25° C. for 5 minutes. Viscosity is then measured using a stress-ramp procedure common on these types of machines using a logarithmic stress ramp from 20 to 2000 Pa at a rate of 60 seconds per decade (2 minute ramp test), with 20 measurements points per decade. The starting and ending stress is sufficient to induce flow and reach a shear rate of at least 10 sec−1. Viscosity is recorded and the data fitted to a power law model using Equation 1. Only points between 0.001 sec−1 and 40 seconds-1 are to be used in the power law fit. The viscosity at 1.0 sec−1 is calculated from Equation 1. One should carefully watch the sample during the test so that when the material is ejected from under the plate, the method is stopped.

Viscosities are recorded and the data fit to a power law with the following Equation 1:

η=κ·γ(dot)(ⁿ⁻¹⁾

where η=viscosity, κ is the consistency and γ(dot) is the shear rate, and n is the shear index. The viscosity at 1 sec−1 is then calculated using the calculated values of κ and n from the fitted data.

Polymeric Stabilizers Viscosity Test:

Polymeric stabilizer phase is formed using the ratio of stabilizer to water that will be found in the particular formulation of interest. For example, if the formulation contains 3 parts polymeric stabilizers and 72 parts water, the ratio will be 1:24. The polymer is hydrated in the water phase at the appropriate ratio. The method of hydration will vary depending upon the polymer type, and may require high shear, heating, and/or neutralization. In any event, the polymer should be properly hydrated according to manufacturer's instructions. Once the polymer is fully hydrated, the system is allowed to sit at room temperature for at least 24 hours. After the resting period, the viscosity of the stabilizer phase is measured with a Brookfield or similar viscometer using a cone and plate (Spindle 41 for a Brookfield model DV II+) geometry at 1 sec−1 and 25 C. 2 ml of the product is placed in the cup of the viscometer and attached to the unit. The rotation is started and after 2 minutes the viscosity is recorded.

Gel-Network Stability Test:

The gel-network phase is formed using the ratio of gel-network materials (non-ionic surfactant, hydrophobic structuring agent) to water as will be found in the particular formulation. For example, if the formulation contains 3 total parts gel-network former and 72 parts water, the ratio will be 1:24. The water is heated, the gel-network materials added and when these materials have dissolved completely (time and temperature will depend upon the materials) the mixture is allowed to cool to room temperature. If after 5 days the mixture has separated (as can be determined by visual inspection of the mixture), then the ratio of water to gel-network former falls within the preferred range.

Lather Volume

Lather volume of a wet skin treatment composition can be measured using a graduated cylinder and a tumbling apparatus. A 1,000 ml graduated cylinder is chosen which is marked in 10 ml increments and has a height of 14.5 inches at the 1,000 ml mark from the inside of its base (for example, Pyrex No. 2982). Distilled water (100 grams at 23° C.) is added to the graduated cylinder. The cylinder is clamped in a rotating device, which clamps the cylinder with an axis of rotation that transects the center of the graduated cylinder. One gram of the total wet skin treatment composition is added into the graduated cylinder and the cylinder is capped. The cylinder is rotated at a rate of 10 revolutions in about 20 seconds, and stopped in a vertical position to complete the first rotation sequence. A timer is set to allow 30 seconds for the lather thus generated to drain. After 30 seconds of such drainage, the first lather volume is measured to the nearest 10 ml mark by recording the lather height in ml up from the base (including any water that has drained to the bottom on top of which the lather is floating).

If the top surface of the lather is uneven, the lowest height at which it is possible to see halfway across the graduated cylinder is the first lather volume (ml). If the lather is so coarse that a single or only a few foam cells (“bubbles”) reach across the entire cylinder, the height at which at least 10 foam cells are required to fill the space is the first lather volume, also in ml up from the base. Foam cells larger than one inch in any dimension, no matter where they occur, are designated as unfilled air instead of lather. Foam that collects on the top of the graduated cylinder but does not drain is also incorporated in the measurement if the foam on the top is in its own continuous layer, by adding the ml of foam collected there using a ruler to measure thickness of the layer, to the ml of foam measured up from the base. The maximum foam height is 1,000 ml (even if the total foam height exceeds the 1,000 ml mark on the graduated cylinder). One minute after the first rotation is completed, a second rotation sequence is commenced which is identical in speed and duration to the first rotation sequence. The second lather volume is recorded in the same manner as the first, after the same 30 seconds of drainage time. A third sequence is completed and the third lather volume is measured in the same manner, with the same pause between each for drainage and taking the measurement.

The lather result after each sequence is added together and the Total Lather Volume determined as the sum of the three measurements, in ml. The Flash Lather Volume is the result after the first rotation sequence only, in ml, i.e., the first lather volume.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total cleansing, treatment compositions, unless otherwise specified.

| | Ex. | Ex. | Ex. | Ex. | Ex. | Ex. | Ex. | Ex. | Ex. |
|---|---|---|---|---|---|---|---|---|---|
| Ingredient | 1 | 2 | 3 | 4 | 5 | 6 | 7 | 8 | 9 |
| I. Aqueous Phase | wt | wt | wt | wt | wt | wt | wt | wt | wt |
| Composition | % | % | % | % | % | % | % | % | % |
| Hydroxypropyl Starch | 3.5 | 4.0 | 3.0 | 4.0 | 3.5 | 3.5 | 4.0 | 4.0 | 3.5 |
| Phosphate (Structure XL from | | | | | | | | | |
| National Starch) | | | | | | | | | |
| Emulsifying Wax NF (Polawax | 2.75 | 3.0 | 2.5 | 3.0 | 2.75 | | 3.0 | 3.0 | 1 |
| from Croda) | | | | | | | | | |
| Tween 60 (Polysorbate-60 from | | | | | | 0.5 | | | |
| ISP) | | | | | | | | | |
| Cetyl Alcohol | | | | | | 0.4 | | | |
| Stearyl Alcohol | | | | | | 0.4 | | | |
| Fragrance | 1.0 | 1.0 | 1.0 | 1.0 | 1.0 | 1.0 | 1.0 | 1.0 | 1.0 |
| Preservatives | 0.8 | 0.8 | 0.8 | 0.8 | 0.8 | 0.8 | 0.8 | 0.8 | 0.8 |
| Water | Q.S. | Q.S. | Q.S. | Q.S. | Q.S. | Q.S. | Q.S. | Q.S. | Q.S. |
| Petrolatum (Superwhite | 15 | 15 | 30 | 25 | 20 | 20 | 15 | | 35 |
| Protopet from WITCO) | | | | | | | | | |
| Mineral Oil (Hydrobrite 1000 | | | | | 5 | | | | |
| PO White MO from WITCO) | | | | | | | | | |
| Jojoba Oil (Lipovol J from | | | | 5 | | | | | |
| Lipo) | | | | | | | | | |
| Silicone Fluid (50 cstk from | | | | | | | 2 | | |
| Dow Corning) | | | | | | | | | |
| Gelled Mineral Oil (Versagel | | | | | | | | 15 | |
| M750 from Penreco) | | | | | | | | | |

The personal care composition of Example 1-9 can be prepared by conventional 5 formulation and mixing techniques. One such example is shown below, although a variety of orders of addition can be used to formulate useable products.

Prepare the aqueous phase composition by first dispersing the hydroxypropyl starch phosphate in water. Add gel network phase (emulsifying wax or tween 80/cetyl alcohol/stearyl alcohol blend) and heat to 160 F. Place mixing vessel in a water bath to cool to under 100 F. Add fragrance.

Premix all lipids at 160 F. Add to the aqueous phase (<80 F) with increased agitation. (In the case of examples with multiple lipids, the lipids can be premixed or not, depending upon the desired outcome.) Add preservatives and agitate until product is smooth.

specified.

| | Ex. | Ex. | Ex. |
|---|---|---|---|
| | 10 | 11 | 12 |
| Ingredient | wt | wt | wt |
| I. Aqueous Phase Composition | % | % | % |
| Sepigel 305 from Seppic | | 0.5 | |
| Hydroxypropyl Starch Phosphate | 3.5 | 2.5 | 3.5 |
| (Structure XL from National | | | |
| Starch) | | | |
| Emulsifying Wax NF (Polawax | 3.0 | 2.25 | 3.0 |
| from Croda) | | | |
| Fragrance | 1.0 | 1.0 | 1.0 |
| Preservatives | 0.8 | 0.8 | 0.8 |
| Water | Q.S. | Q.S. | Q.S. |
| Petrolatum (Superwhite Protopet | 20 | | |
| from WITCO) | | | |
| Mineral Oil (Hydrobrite | | | 5 |
| 1000 PO White MO | | | |
| from WITCO) | | | |
| G-2180 Petrolatum from | | 25 | 20 |
| Crompton | | | |
| Gelled Mineral Oil (Versagel | 5 | | 5 |
| M750 from Penreco) | | | |

The personal care composition of Example 10-12 can be prepared by conventional 10 formulation and mixing techniques. One such example is shown below, although a variety of orders of addition can be used to formulate useable products.

First, prepare the aqueous phase composition by dispersing the hydroxypropyl starch phosphate in water. Add gel network phase (emulsifying wax or tween 80/cetyl alcohol/stearyl alcohol blend) and heat to 160 F. Place mixing vessel in a water bath to cool to under 100 F. Add fragrance.

Add the lipid(s) (preheated to 160 F) to the aqueous phase (<80 F) with increased agitation. (In the case of examples with multiple lipids, the lipids can be premixed or not, depending upon the desired outcome.) Add preservatives and agitate until product is smooth.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An oil-containing composition comprising:
a) an aqueous phase;
b) from about 1 to about 80 weight percent of a structured oil phase;
c) a gel-network phase comprising a non-ionic hydrophilic surfactant and a hydrophobic structuring agent; and
d) an aqueous phase stability agent
wherein said structured oil phase has a shear index of less than 0.8.

2. The composition of claim 1, comprising at least 10 weight percent of said structured oil phase.

3. The composition of claim 1, comprising at least 20 weight percent of said structured oil phase.

4. The composition of claim 1, wherein said structured oil phase is petrolatum.

5. The composition of claim 1, wherein said gel-network phase is present in an amount from about 1 to about 20 weight percent of said composition.

6. The composition of claim 1, wherein said gel-network phase is present in an amount from about 0.5 to about 20 weight percent of said composition.

7. The composition of claim 1, wherein said gel-network phase is present in an amount from about 1 to about 10 weight percent of said composition.

8. The composition of claim 1, wherein said aqueous phase stability agent is a modified starch.

9. A method of depositing oils on a substrate comprising applying an oil-containing composition to a substrate, said oil-containing composition comprising:
a) an aqueous phase;
b) from about 1 to about 80 weight percent of a structured oil phase;
c) a gel-network phase comprising a non-ionic hydrophilic surfactant and a hydrophobic structuring agent; and
d) an aqueous phase stability agent
wherein said structured oil phase has a shear index of less than 0.8.

10. The method of claim 9, wherein said oil-containing composition comprises at least 10 weight percent of said structured oil phase.

11. The method of claim 9, wherein said oil-containing composition comprises at least 20 weight percent of said structured oil phase.

12. The method of claim 9, wherein said gel-network phase is present in an amount from about 0.5 to about 20 weight percent of said composition.

13. The method of claim 9, wherein said gel-network phase is present in an amount from about 1 to about 10 weight percent of said composition.

14. The method of claim 9, wherein said structured oil phase is petrolatum.

15. The method of claim 9, wherein said aqueous phase stability agent is a modified starch.
